(19) United States
(12) Patent Application Publication
(10) Pub. No.: US 2024/0000440 A1
(43) Pub. Date: Jan. 4, 2024

(54) PROCESSES FOR USING TEMPERATURE MEASUREMENTS TO DETERMINE HEALTH METRICS

(71) Applicant: ORPYX MEDICAL TECHNOLOGIES INC., CALGARY (CA)

(72) Inventors: JULIA BREANNE EVERETT, CALGARY (CA); TRAVIS MICHAEL STEVENS, CALGARY (CA)

(73) Assignee: ORPYX MEDICAL TECHNOLOGIES INC., CALGARY (CA)

(21) Appl. No.: 17/854,004

(22) Filed: Jun. 30, 2022

(51) Int. Cl. A61B 10/00; G16H 40/67; G16H 50/30; A61B 5/01; A61B 5/00

(52) U.S. Cl. CPC A61B 10/0012; G16H 40/67; G16H 50/30; A61B 5/01; A61B 5/6807; A61B 2010/0019

(57) ABSTRACT

A process for using temperature measurements to determine health metrics includes: a) using a first temperature sensor of a wearable device to obtain a first set of measurements at a first location on a user's body over a time period; b) using a second temperature sensor of the wearable device to obtain a second set of measurements at a second location on the user's body over the time period; c) processing the measurements to identify changes in temperature over the time period, determining a first health metric based on the changes, and outputting an indication of the first health metric; and d) processing the measurements to compare the temperature at the first location at a given time point to the temperature at the second location at the given time point, determining a second health metric based on the comparison, and outputting an indication of the second health metric.

### FIELD

This document relates to health and wellness. More specifically, this document relates to processes for using temperature measurements to determine health metrics. Such processes may relate to COVID-19.

### BACKGROUND

U.S. Pat. No. 10,004,428 (Everett et al.) discloses a sensor-based quantification and analysis system including an input device including a plurality of sensors that generate an input based on a force. The input device also includes a transmission device that transmits force information based on the input. The system also includes an output device that receives the force information. A processing unit selects, for each of the plurality of sensors, one of a plurality of levels of a likelihood of tissue damage based on the force and a predetermined time period. Further, the output device includes a display that presents or logs the one of the plurality of levels of the likelihood of tissue damage for each of the plurality of sensors.

### SUMMARY

The following summary is intended to introduce the reader to various aspects of the detailed description, but not to define or delimit any invention.

Processes for using temperature measurements to determine health metrics are disclosed.

According to some aspects, a process for using temperature measurements to determine health metrics includes: a) while a wearable device is worn by a user, using a first temperature sensor of the wearable device to obtain a measurement at a first location on the user's body; b) serially repeating step a) to obtain a first set of measurements over a time period; c) using a second temperature sensor of the wearable device to obtain a measurement at a second location on the user's body; d) serially repeating step c) to obtain a second set of measurements over the time period; e) processing at least some measurements of the first set of measurements and/or at least some measurements of the second set of measurements to identify changes in a body temperature of the user over the time period, determining at least one first health metric based on the changes, and outputting an indication of the first health metric; and f) for each of a plurality of time-points within the time period, processing the measurement of the first set taken at the time-point and the measurement of the second set taken at the time-point to compare the temperature at the first location at the time point to the temperature at the second location at the time point, determining at least one second health metric based on the comparison, and outputting an indication of the second health metric.

In some examples, the first health metric includes a phase of a menstrual cycle of the user. The time period can be a multi-day period, and step e) can include: i) for each day in the multi-day period, determining a daily average temperature by processing a subset of the first set of measurements and a subset of the second set of measurements, wherein the subset of the first set of measurements and the subset of the second set of measurements are taken from a daily window; and ii) identifying changes in the daily average temperature over the multi-day period to identify the phase of the menstrual cycle of the user, and outputting an indication of the phase of the menstrual cycle. Step ii) can include identifying an ovulation phase and/or a menstruation phase in the menstrual cycle of the user. The process can further include predicting a future phase of a future menstrual cycle, and outputting an indication of the prediction. The process can further include: i) using the wearable device to identify a time period during which the user is at rest; and ii) setting the daily window as the time period during which the user is at rest.

In some examples, step e) can include: i) for each of the plurality of time-points within the time period, determining a time-point average temperature by processing the measurement of the first set at the time-point and the measurement of the second set at the time-point; and ii) identifying changes in the time-point average temperatures over the time period to identify the first health metric, and outputting the indication of the first health metric. The first health metric can include a period of physical activity for the user, and step ii) can include identifying an increase in the time-point average temperatures over the time period to identify the period of physical activity for the user. The first health metric can include a fever state for the user, and step ii) can include identifying whether the time-point average temperature has exceeded a fever threshold over the time period to identify the fever state of the user.

In some examples, the second health metric includes a risk of tissue ulceration at the first location and/or the second location. Step f. can include identifying whether the temperature at the first location differs from the temperature at the second location by a threshold.

In some examples, the wearable device includes an insole, the first location is a first plantar location on a first foot of the user, and the second location is a second plantar location on the first foot of the user.

In some examples, the wearable device includes a pair of insoles, the first location is a first plantar location on a first foot of the user, and the second location is a second plantar location on a second foot of the user.

According to some aspects, a process for using temperature measurements to determine health metrics includes: a) while a wearable device is worn by a user, using a first temperature sensor of the wearable device to obtain a measurement at a first location on the user's body; b) serially repeating step a. to obtain a first set of measurements over a time period; c) using a second temperature sensor of the wearable device to obtain a measurement at a second location on the user's body; d) serially repeating step c. to obtain a second set of measurements over the time period; e) for each of a plurality of time-points within the time period, determining a time-point average temperature by processing the measurement of the first set at the time-point and the measurement of the second set at the time-point; f) identifying changes in the time-point average temperatures over the time period to identify at least one first health metric for the user, and outputting an indication of the first health metric; and g) for each of the plurality of time-points within the time period, processing the measurement of the first set taken at the time-point and the measurement of the second set taken at the time-point to compare the temperature at the first location at the time point to the temperature at the second location at the time point, determining at least one second health metric based on the comparison, and outputting an indication of the second health metric.

In some examples, the first health metric includes a period of physical activity for the user, and step f) includes identifying an increase in the time-point average temperatures over the time period to identify the period of physical activity for the user.

In some examples, the first health metric includes a fever state for the user, and step f) includes identifying whether the time-point average temperatures have exceeded a fever threshold over the time period to identify the fever state of the user. The process may further include instructing the user to take a COVID-19 test if the time-point average temperatures have exceeded a fever threshold over the time period.

In some examples, the second health metric includes a risk of tissue ulceration at the first location and/or the second location. Step g) can include identifying whether the temperature at the first location differs from the temperature at the second location by a threshold.

In some examples, the wearable device includes an insole, the first location is a first plantar location on a first foot of the user, and the second location is a second plantar location on the first foot of the user.

In some examples, the wearable device includes a pair of insoles, the first location is a first plantar location on a first foot of the user, and the second location is a second plantar location on a second foot of the user.

According to some aspects, a process for using temperature measurements to determine health metrics includes: a) while a wearable device is worn by a user, using a first temperature sensor of the wearable device to obtain a measurement at a first location on the user's body; b) serially repeating step a. to obtain a first set of measurements over a multi-day time period; c) using a second temperature sensor of the wearable device to obtain a measurement at a second location on the user's body; d) serially repeating step c. to obtain a second set of measurements over the multi-day time period; e) for each day in the multi-day time period, determining a daily average temperature by analyzing a subset of the first set of measurements and a subset of the second set of measurements, wherein the subset of the first set of measurements and the subset of the second set of measurements are taken from a daily window; f) identifying changes in the daily average temperatures over the multi-day period to identify at least one first health metric for user, and outputting an indication of the first health metric; and g) for each of a plurality of time-points within the multi-day period, processing a measurement of the first set taken at the time-point to a measurement of the second set taken the time-point to compare the temperature at the first location at the time point to the temperature at the second location at the time point, determining at least one second health metric based on the comparison, and outputting an indication of second health metric.

In some examples, the first health metric includes a phase of a menstrual cycle of the user. Step f. can include identifying an ovulation phase and/or a menstruation phase in the menstrual cycle of the user. The process can further include predicting a future phase of a future menstrual cycle, and outputting an indication of the prediction. The process can further include: i) using the wearable device to identify a time period during which the user is at rest; and ii) setting the daily window as the time period during which the user is at rest.

In some examples, the second health metric includes a risk of tissue ulceration at the first location and/or the second location. Step g) can include identifying whether the temperature at the first location differs from the temperature at the second location by a threshold.

In some examples, the wearable device includes an insole, the first location is a first plantar location on a first foot of the user, and the second location is a second plantar location on the first foot of the user.

In some examples, the wearable device includes a pair of insoles, the first location is a first plantar location on a first foot of the user, and the second location is a second plantar location on a second foot of the user.

### BRIEF DESCRIPTION OF THE DRAWINGS

The drawings included herewith are for illustrating various examples of articles, processes, and apparatuses of the present specification and are not intended to limit the scope of what is taught in any way. In the drawings:

FIG. 1 is a perspective view of an example wearable device that can be used to take measurements for the determination of health metrics;

FIG. 2 is an exploded view of the wearable device of FIG. 1;

FIG. 3 is a flow chart of a process for processing measurements, such as measurements taken by the wearable device of FIGS. 1 and 2;

FIG. 4 is a schematic view of an example user interface that can be used to display health metrics, such as health metrics determined using the wearable device of FIGS. 1 and 2; and

FIG. 5 is a flow chart of a process for using measurements to determine health metrics.

### DETAILED DESCRIPTION

Various apparatuses or processes or compositions will be described below to provide an example of an embodiment of the claimed subject matter. No embodiment described below limits any claim and any claim may cover processes or apparatuses or compositions that differ from those described below. The claims are not limited to apparatuses or processes or compositions having all of the features of any one apparatus or process or composition described below or to features common to multiple or all of the apparatuses or processes or compositions described below. It is possible that an apparatus or process or composition described below is not an embodiment of any exclusive right granted by issuance of this patent application. Any subject matter described below and for which an exclusive right is not granted by issuance of this patent application may be the subject matter of another protective instrument, for example, a continuing patent application, and the applicants, inventors or owners do not intend to abandon, disclaim or dedicate to the public any such subject matter by its disclosure in this document.

For simplicity and clarity of illustration, reference numerals may be repeated among the figures to indicate corresponding or analogous elements. In addition, numerous specific details are set forth in order to provide a thorough understanding of the subject matter described herein. However, it will be understood by those of ordinary skill in the art that the subject matter described herein may be practiced without these specific details. In other instances, well-known methods, procedures and components have not been described in detail so as not to obscure the subject matter described herein. The description is not to be considered as limiting the scope of the subject matter described herein.

The terms “coupled” or “coupling” or as used herein can have several different meanings depending on the context in which these terms are used. For example, the terms coupled or coupling can have a mechanical, electrical or communicative connotation. For example, as used herein, the terms coupled or coupling can indicate that two elements or devices are directly connected to one another or connected to one another through one or more intermediate elements or devices via an electrical element, electrical signal, or a mechanical element depending on the particular context.

As used herein, the wording “and/or” is intended to represent an inclusive-or. That is, “X and/or Y” is intended to mean X or Y or both, for example. As a further example, “X, Y, and/or Z” is intended to mean X or Y or Z or any combination thereof. Furthermore, the wording “at least one of A and B” is intended to mean only A, only B, or A and B.

Terms of degree such as “substantially”, “about”, and “approximately” as used herein mean a reasonable amount of deviation of the modified term such that the end result is not significantly changed. These terms of degree may also be construed as including a deviation of the modified term if this deviation would not negate the meaning of the term it modifies.

Any recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about”, which means a variation of up to a certain amount of the number to which reference is being made if the end result is not significantly changed.

The systems, processes, and devices described herein may be implemented as a combination of hardware or software. In some cases, the systems, processes, and devices described herein may be implemented, at least in part, by using one or more computer programs, executing on one or more programmable devices including at least one processing element, and a data storage element (including volatile and non-volatile memory and/or storage elements). These devices may also have at least one input device (e.g. a pushbutton keyboard, mouse, a touchscreen, and the like), and at least one output device (e.g. a display screen, a printer, a wireless radio, and the like) depending on the nature of the device.

Some elements that are used to implement at least part of the systems, processes, and devices described herein may be implemented via software that is written in a high-level procedural language such as object-oriented programming. Accordingly, the program code may be written in any suitable programming language such as Python or C, for example. Alternatively, or in addition thereto, some of these elements implemented via software may be written in assembly language, machine language or firmware as needed. In either case, the language may be a compiled or interpreted language.

At least some of these software programs may be stored on a storage media (e.g. a computer readable medium such as, but not limited to, ROM, magnetic disk, optical disc) or a device that is readable by a general or special purpose programmable device. The software program code, when read by the programmable device, configures the programmable device to operate in a new, specific and predefined manner in order to perform at least one of the processes described herein.

Furthermore, at least some of the programs associated with the systems and processes described herein may be capable of being distributed in a computer program product including a computer readable medium that bears computer usable instructions for one or more processors. The medium may be provided in various forms, including non-transitory forms such as, but not limited to, one or more diskettes, compact disks, tapes, chips, and magnetic and electronic storage. Alternatively, the medium may be transitory in nature such as, but not limited to, wire-line transmissions, satellite transmissions, internet transmissions (e.g. downloads), media, digital and analog signals, and the like. The computer useable instructions may also be in various formats, including compiled and non-compiled code.

Generally disclosed herein are processes for using temperature sensors to determine health metrics. In general, in the processes disclosed herein, a wearable device is used to determine a user's body temperature. In particular, the wearable device may include multiple (i.e. at least two) temperature sensors. The temperature sensors may be used to take measurements at multiple (i.e. at least two) locations on the user's body, and to take the measurements at the different locations over a period of time (e.g. continuously, intermittently, or sporadically). The measurements can then be processed in order to determine multiple (i.e. at least two) health metrics. Particularly, a first health metric can be determined by identifying changes in the user's body temperature over time. For example, by identifying changes in the user's body temperature over time, a phase of the user's menstrual cycle can be determined (e.g. ovulation or menstruation can be predicted), fertility can be tracked, a fever in the user can be identified (e.g. for the purpose of identifying COVID-19 infection), a period of physical activity for the user can be identified, or an intensity of physical activity can be identified. Further, a second health metric can be determined by comparing the user's body temperature at one location to the user's body temperature at one or more other locations. For example, by comparing the body temperature at different locations, a risk of tissue ulceration (e.g. a risk of a diabetic foot ulcer) can be identified.

As used herein, the term ‘health metric’ refers to any information relating to a user's body, including but not limited to information relating to a change in a physiological state, a response to a medication, illness (including physical and mental illness, and acute and chronic illness), wellness, physical activity, tissue damage, habits, fertility, infection, and a phase of a menstrual cycle. Specific non-limiting examples of health metrics include a fever state of the user (e.g. psychogenic fever), hormonal fluctuations, hypothermia, vascular status of a limb (or portion thereof), wound healing, state of shock, body temperature (e.g. basal body temperature, or body temperature for the purpose of assessing thermoregulation issues), an ovulation phase of the user's menstrual cycle, a menstrual phase of the user's menstrual cycle, a risk of tissue ulceration, a period of physical activity for the user, sleep habits, area specific inflammation, and pregnancy. Health metrics may relate to a current state of the user's body, a past state of a user's body, or a future state of the user's body. For example, determining a health metric may include identifying that a user is ovulating, identifying that a user has ovulated, or predicting that a user will ovulate within a given time frame. For further example, determining a health metric may include identifying that a user has a diabetic foot ulcer, or predicting that a user will develop a diabetic foot ulcer if the user does not rest their foot.

As used herein, the term ‘wearable device’ refers to any device that is worn in sufficiently close proximity to a user's body to allow for temperature sensors of the wearable device to be used to determine or estimate or approximate a temperature of the user's body (e.g. the skin temperature at a location proximate temperature sensor). A wearable device can be in direct contact with a user's skin, or can be spaced from the user's skin. For example, a wearable device may be in the form of a thigh band worn directly on the skin of the user's thigh. Alternatively, a wearable device can be in the form of an insole, which may in some examples be separated from the user's skin by a sock, and may in some examples be worn directly against the user's skin. Specific non-limiting examples of wearable devices include wearable textiles, socks, upper-body garments, lower-body garments, watches, rings, necklaces, head garments (e.g. hats or headbands), shoes, insoles, bandages, adhesive nodes, thigh bands, hearing aids, ear pods, and undergarments. Preferably, the wearable devices described herein may be worn in areas that are prone to ulceration, such as the sole of the foot.

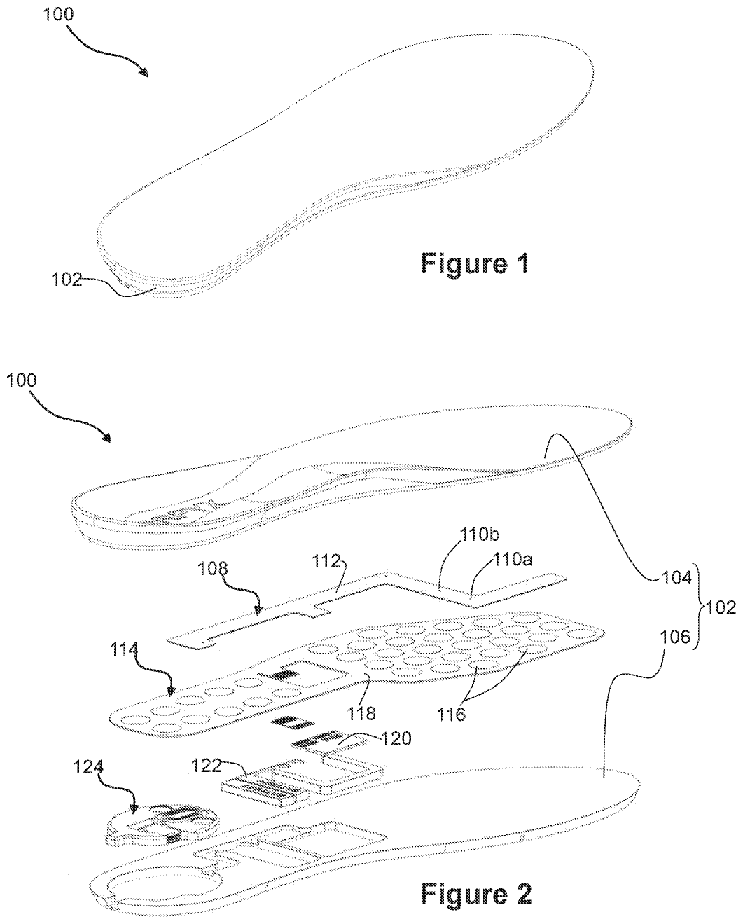

Referring now to FIGS. 1 and 2, a first example of a wearable device is shown. In the example shown, the wearable device is an insole 100, which may be inserted into a user's shoe (not shown) and worn against the sole of the user's foot (not shown). As mentioned above, the insole 100 may be worn in direct contact with the user's skin, or may be spaced from the user's skin (e.g. by a sock). Insoles usable in the processes described herein may be of a variety of configurations, of which insole 100 is but one example. For example, the insoles may be those available from Orpyx Medical Technologies Inc. (Calgary, Canada) under the brand name Orpyx SI®. For further example, the insoles may be those described in U.S. Pat. No. 10,004,428 (Everett et al.), International Patent Application Publication No. WO/2021/092676 A1 (Everett et al.), U.S. patent application Ser. No. 17/525,092 (Stevens et al.), and/or U.S. Patent Application No. 63/290,247 (Stevens et al.). Each of the aforementioned documents is incorporated herein by reference in its entirety.

Referring first to FIG. 1, in the example shown, the insole 100 includes an insole bulk 102, which may be made up of one or more layers such as a cushion layer, a support layer, a gel layer, an anti-odor layer, a thermal insulation layer, and/or a foam layer. In the example shown, the insole 100 is in the form of an orthotic that is custom manufactured for a user. For example, the user's foot may be assessed (e.g. by a podiatrist, optionally using plaster casting or 3D scanning), and the insole bulk 102 may be custom fashioned based on the assessment, for example in order to support the user's foot, improve foot function, relieve pain, and/or relieve pressure. In the example shown, the insole bulk 102 includes a top layer 104 and a base layer 106 (shown in FIG. 2). The top layer 104 may in turn include multiple sub-layers, such as an upper finishing layer (not shown), a middle comfort layer (not shown), and a contoured layer (not shown). Likewise, the base layer may include multiple sub-layers (not shown).

In other examples, the insole may be another type of insole, such as a generic insole (e.g. a comfort insole, a flat insole, an athletic insole, a shock-absorbing insole, or a gel insole). Furthermore, the insole may in some examples be integral with a shoe. The insole may be available in different sizes.

Referring to FIG. 2, in the example shown, a temperature sensor array 108 is embedded in the insole bulk 102. The temperature sensor array 108 includes a plurality of temperature sensors 110 (i.e. a first temperature sensor 110a, a second temperature sensor 110b, and so on, only two of which are labelled) printed on flexible polymer film 112. The temperature sensors 110 are spaced apart such that in use, they obtain measurements at different locations on the body. More specifically, the first temperature sensor 110a is positioned to obtain a measurement at a first location on the sole of the user's foot, the second temperature sensor 110b is positioned to obtain a measurement at a second location on the sole of the user's foot, and so on. At least one of the temperature sensors 110 may be positioned such that in use, it is adjacent an area known to be at high risk for pressure ulcer formation in diabetic patients, such as the first metatarsal phalangeal (MTP) joint. The temperature sensors 110 may be, for example, multilayer chip thermistors, and may have an accuracy of at least 0.1 degrees Celsius. The temperature sensors 110 may be configured to serially obtain measurements, for example continuously at a frequency of about or at least 4 Hz, or intermittently at regular intervals, or sporadically.

Referring still to FIG. 2, in the example shown, the insole 100 further includes a pressure sensor 114 array embedded in the insole bulk 102. The pressure sensor array 114 includes a plurality of pressure sensors 116 (only two of which are labelled) printed on flexible polymer film 118.

Referring still to FIG. 2, the insole 100 further includes a processor 120 that receives measurements from the temperature sensors 110 and the pressure sensors 116, and processes the measurements to yield temperature information and pressure information (as described in further detail below). The temperature information and/or pressure information can include one or more health metrics. Alternatively, the temperature information and/or pressure information can include information that will be further processed (e.g. by a secondary device as described below) to yield one or more health metrics.

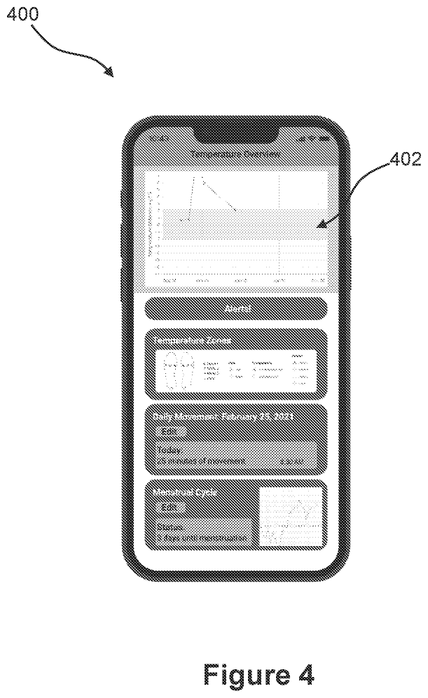

The temperature information and pressure information can then be transmitted out of the insole 100 to one or more secondary devices, such as but not limited to a smartphone 400 (shown in FIG. 4), a smart watch, a tablet, a computer (e.g. a laptop or desktop computer), a wall hub, a purpose-built device, and/or a cloud server. This transmission can occur via a wired connection (not shown) or a wireless connection (e.g. the insole 100 can include a wireless transmission node (not shown) that is in communication with the processor 120). The secondary device(s) optionally can further process the temperature information and/or pressure information to yield one or more health metrics. The secondary device(s) can then display the health metrics.

Referring still to FIG. 2, a pair of batteries 122 are further embedded in the insole bulk 102, for providing energy to the components of the insole 100. The batteries 122 can be charged by a wireless charging assembly, which includes a wireless charging transmitter (not shown) and a wireless charging receiver 124 that is embedded in the insole bulk 102.

Various additional sensors may be included in the insole 100. Such sensors may include one or more of a GPS sensor, a heart rate sensor, a respiratory rate sensor, a blood pressure sensor, a blood oxygen saturation sensor, a blood flow sensor, a blood or environmental content quantification sensor (e.g. glucose, electrolytes, minerals, oxygen, carbon dioxide, carbon monoxide, HbA1C, Ethanol, protein, lipid, carbohydrate, cortisol, lactate, pH, pro- and anti-inflammatory markers, MMPs, growth factors, bacterial content), a hydration status/tissue turgor sensor, a joint position sensor, a gait analysis sensor (including supination and pronation), a device breakdown sensor, a pedometer, an accelerometer, a velocity sensor, a calorimetry sensor, a centre of gravity sensor, a centre of foot position sensor, a friction sensor, a traction sensor, a contact area sensor, a connectivity/insulation sensor, an EEG sensor, an inertial measurement unit (IMU), a barometer, an altimeter, and/or an ECG sensor.

In some examples, the insole 100 may include one or more additional sensors that are susceptible to mechanical and/or electrical changes due to temperature changes (e.g. strain gauge sensors). Thus, temperature changes may interfere with measurements from these additional sensors. Optionally, the measurements from the temperature sensors may be used to adjust the values from these additional sensors, in order to improve the accuracy of readings from these additional sensors.

Optionally, the insole 100 may include one or more stimulators (not shown) for providing feedback to the user. The stimulator(s) may provide a tactile (e.g. vibratory), audible, or visual stimulus to the user. For example, the insole 100 may include thermochromic materials to visually indicate temperature. For further example, if there is a relatively hot zone on the ball of the foot, the insole may turn red, to indicate to the user to rest the foot. The feedback may encourage or direct the user to take a specified action (e.g. red may encourage the user to rest the foot). The stimulators may be triggered by the sensor values or first or second health metrics, or they may be triggered by a separate user (e.g. a clinician or nurse), who reviews the data live or post-hoc.

The components of the insole 100 may be generally flat, and/or may be nested within pockets of the insole bulk 102, so that in use the user generally does not feel the presence of a foreign object under their foot.

In the example shown, the wearable device is in the form of a single insole 100; however, in alternative examples, the wearable device may be in the form of a pair of insoles (i.e. a left insole and a right insole, which can be worn concurrently).

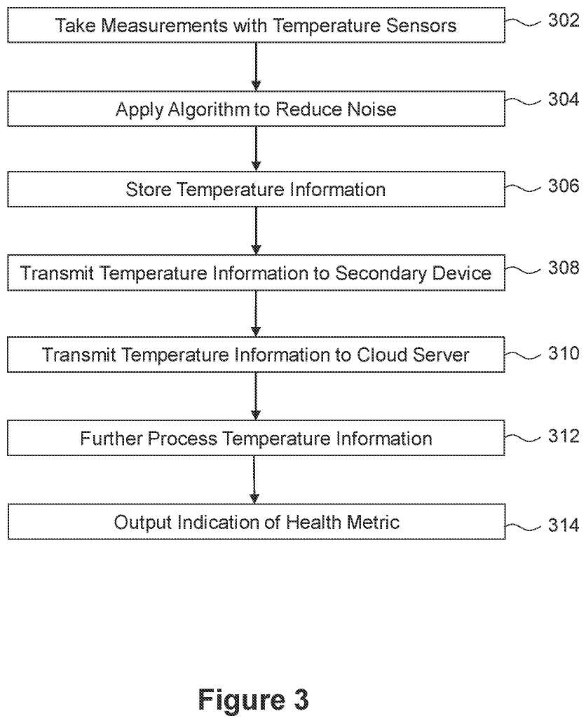

As mentioned above, measurements from the temperature sensors 110 can be processed within the insole 100 to yield temperature information, which may be further processed by one or more secondary devices. More specifically, in one particular example shown in FIG. 3, the temperature sensors may take measurements (Step 302), which may include raw data such as resistance data, capacitance data, and/or other raw data. This raw data is sent to the processor 120, which may apply an algorithm for reducing data noise (Step 304), and/or may scale and/or calibrate the raw data into meaningful units. This algorithm may, for example, determine average (or median or mode) values over a designated period or number of data points. The temperature information described above may include these average (or median or mode) values. The temperature information may then optionally be stored in a memory on the insole (Step 306). At an appropriate time (e.g. when placed in wired communication with a secondary device or when a wireless connection to a secondary device is available), the temperature information be transmitted to a secondary device (Step 308), which may be in the form of a relay device (e.g. a smart phone or wall hub). The relay device may optionally store the temperature information, and may transmit the temperature information to a cloud server (Step 310). The cloud server may store the temperature information and may further process the temperature information (Step 312). Particularly, the cloud server may allow for more complex processing of the temperature information. For example, the cloud server may apply an algorithm for separating whole-foot temperature averages and individual temperature sensor values. Based on the processing, one or more health metrics may then be output (step 314). That is, one or more health metrics may be displayed or communicated to the user and/or a third party, such as a healthcare practitioner. The health metrics may be displayed, for example, via a mobile application or a dashboard of a web-based application. FIG. 4 shows a smart phone 400 that includes a user interface 402 of a mobile application, in which a set of health metrics are displayed.

Measurements from the pressure sensors 116 may be handled in a similar fashion to the measurements from the temperature sensors 110. That is, measurements from the pressure sensors 116 may include raw data such as resistance data, capacitance data, and/or other raw data. This raw data may be sent to the processor 120, which may apply an algorithm for reducing data noise, and/or for scaling/calibrating the data, to yield pressure information. The pressure information may then be transmitted to a relay device, which may store the pressure information and transmit the pressure information to a cloud server. The cloud server may store the pressure information and/or allow for more complex processing of the pressure information. For example, the cloud server may apply an algorithm for determining high pressure zones within the insole 100.

In an alternative example, the raw data may be transmitted to the secondary device for processing (i.e. it is possible for no processing or for minimal processing to occur within the insole 100). In a further alternative example, all or most processing may occur within the insole 100.

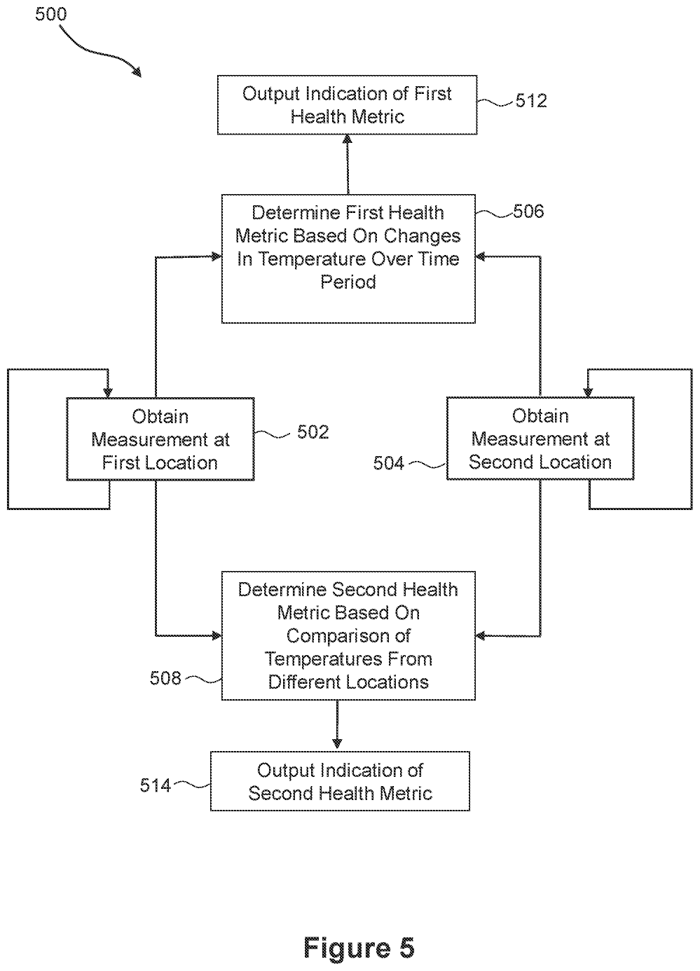

Referring now to FIG. 5, a process 500 for using measurements (e.g. from temperature sensors 110) to determine health metrics will be described. The process will be described with reference to the insole 100 shown in FIGS. 1 and 2 and the communication process shown in FIG. 3; however, as mentioned above, the process can be carried out with other wearable devices, and using other communication processes. Furthermore, for clarity, terms such as “first”, “next”, and “then” will be used to describe the sequence of the steps of the process; however, unless expressly stated as such, the process is not limited to any particular sequence of the steps.

At the start of the process, while the insole 100 is worn by the user, the temperature sensors 110 may be used to obtain measurements over the sole of the user's foot. Particularly, the first temperature sensor 110a may be used to obtain a measurement at a first location (e.g. at the first MTP joint) (Step 502), the second temperature sensor 110b may be used to obtain a measurement at a second location (e.g. at the second MTP joint) (Step 504), and so on with any further temperature sensors 110. Steps 502 and 504 may be repeated serially. That is, the first temperature sensor 110a may serially obtain measurements at the first location, to obtain a first set of measurements over a time period, and the second temperature sensor 110b may serially obtain measurements at the second location, to obtain a second set of measurements over the time period, and so on.

The time period may be, for example, a multi-minute time period, or a multi-hour time period, or a multi-day time period, or a multi-week time period, or a multi-month time period, or a multi-year time period. The insole 100 can be, but need not be, worn continuously over the time period. For example, in the case of a multi-day (or longer) time period, the user may wear the insole 100 during the day, and remove the insole nightly to charge the batteries 122.

Each temperature sensor 110 can optionally take measurements at identical or substantially identical time points over the time period. For example, each temperature sensor 110 may synchronously take a measurement on startup, and may then continue to synchronously take measurements every 0.25 seconds over the time period.

The measurements can then be processed (i.e. by the processor 120 of the insole 100 and/or by the secondary device) in order to determine multiple (i.e. at least two) health metrics. Particularly, at least some measurements of the first set of measurements and/or at least some measurements of the second set of measurements can be processed to identify changes in the body temperature of the user over the time period, and a first health metric can be determined based on the changes (Step 506). Further, for individual time points within the time period, a measurement from the first set of measurements and a measurement from the second set of measurements (where both measurements are taken at the same time point) can be processed to compare the temperature at the first location to the temperature at the second location and to determine at least one second health metric based on the comparison (Step 508).

For example, with regards to Step 506, for each respective time-point in the time period (or for a subset of time-points within the time period), the respective measurements from some or all of the temperature sensors can be averaged, to determine a “time-point average temperature”. Optionally, in order to improve accuracy, outlier measurements can be omitted. For example, if a measurement from a given temperature sensor 110 indicates a difference of more than a predetermined amount (e.g. 1.5 degrees Celsius or 3 degrees Celsius) from the other temperature sensors 110, the measurement from the given temperature sensor 110 may be omitted in the determination of the time-point average. Furthermore, interpolation and/or extrapolation may be carried out in order to estimate the temperature at locations between sensors or spaced from sensors. An example of such interpolation and extrapolation is described in U.S. Provisional Patent Application No. 63/282,234 filed on Nov. 23, 2021, which is incorporated herein by reference in its entirety. In addition to spatial interpolation, temporal interpolation may also be carried out. For example, an algorithm could be implemented to interpolate the temperature in between measurements to artificially increase the scanning frequency. Changes in the time-point average temperature over the time period can then be identified, to determine a first health metric (or multiple first health metrics). An indication of the first health metric(s) can then be output (step 512). For example, as shown in FIG. 4, the secondary device can display an indication of the first health metric(s).

As a first example of determining a first health metric, a period of physical activity (e.g. exercise or other physical exertion) for the user can be identified. Optionally, the relative intensity of the physical activity can be identified (e.g. walking versus running). This can be done by processing the measurements to determine the time-point average temperatures over the time period, and to identify a relevant increase in the time-point average temperatures, such as a gradual and small increase over a relatively short period (e.g. 1 hour). A subsequent drop in the time-point average temperature can indicate that the physical activity has ceased. Conversely, a relevant decrease in the time-point average temperatures can be identified, such as a gradual and small decrease over a relatively short period (e.g. 1 hour). This may indicate a period of rest and/or recovery.

Optionally, to aid in identifying periods of physical activity, the user may input information, such as a time that exercise began and a time that exercise stopped, the location of the physical activity (e.g. indoors or outdoors), the equipment used (e.g. none versus a treadmill) or another indication of a physical activity (e.g. a distance ran). This information may be input using the secondary device (e.g. via user interface 402, or via a dashboard of a web-based application).

Optionally, measurements from the pressure sensors 116 (or other sensors such as an IMU) may be used to aid in identifying a period of physical activity. For example, high or varying pressure values in combination with an increase in the time point average temperature may indicate that physical activity is occurring. Cyclic pressure values may also indicate that physical activity is occurring (e.g. walking). For further example, increased peak accelerations, as detected by an IMU, may indicate physical activity.

As a second example of determining a first health metric, a fever state of the user can be identified. For example, the measurements can be processed to determine the time-point average temperatures over the time period, to compare the time point average temperatures to a fever threshold (e.g. 38.0 degrees Celsius), and to identify whether the time point average temperatures have exceeded the fever threshold. If the time point average temperatures have exceeded the fever threshold for the duration of the time period or for a predetermined window within the time period (e.g. a 1 hour window), this may indicate that the user has a fever.

In a further example, for each day within the time period, all or a subset of the time-point average temperatures can in turn be averaged, to determine a daily average temperature for each day. The daily average temperatures can then be compared to the fever threshold, to identify whether any of the daily average temperatures have exceeded the fever threshold. If a daily average temperature has exceeded the fever threshold, this can indicate that the user has a fever.

In a further example, a fever state can be determined by identifying patterns in the daily average temperature to set the fever threshold, and then identifying whether the fever threshold has been exceeded. In such cases, the fever threshold may be below what is clinically considered a fever. For example, over the first month of a multi-month time period, the daily average temperature for a male user may be determined and stored, and a normal temperature range for that user may then be identified. The fever threshold can then be set at a predetermined amount (e.g. 0.5 degrees Celsius above the daily average temperature). For example, it may be determined that for the user, the normal range in the daily average temperature is between 36.0 and 36.5 degrees Celsius. A temperature of 37.0 degrees can then be set as the fever threshold. Then, over subsequent months, fever may be detected by identifying situations in which the daily average temperature exceeds the fever threshold (e.g. for the duration of the time period or for a predetermined window within the time period).

In any of the above examples of identifying a fever state for the user, if a fever is identified, the user can be instructed to verify their body temperature using a secondary process (e.g. an oral thermometer), to take a medical test (e.g. a COVID-19 rapid test or PCR test), or to seek medical care. These instructions can be conveyed, for example, via the user interface 402.

In a further example, chronic stress may be identified by monitoring the fever state of the user. This may be achieved by identifying a fever state in the user as described above; however, based on inputs from the user (e.g. inputs related to emotions and/or symptoms, underlying mental health conditions), the fever may be identified as a psychogenic fever.

In a further example, fevers or thermoregulation issues after injury (e.g. brain injuries) may be identified.

As a third example of determining a first health metric, a phase of a menstrual cycle of the user can be identified. This can be, for example, for the purpose of tracking or monitoring fertility.

For example, over a multi-day time period (such as several days or several weeks or several months), a daily average temperature may be determined. In some examples, the daily average temperature may be determined from a subset of the time-point average temperatures for a given day. That is, in order to obtain accurate measurements, it may be beneficial to determine the daily average temperature based on measurements taken when the user has just woken or is at rest (which may include sitting rest or standing rest). Accordingly, the daily average temperature may be determined from the time-point average temperatures from a daily window, where the daily window is selected, for example, as the first 15 minutes of use of the insole 100 for a given day, or an overnight window (e.g. between the hours of midnight and 10:00 am), and/or a window after which the user has rested for more than 30 minutes. Periods of rest can be determined, for example, based on the measurements from the pressure sensors 116, where the measurements indicate that the feet are bearing less than 50% of the body weight of the user. Alternatively, periods of rest can be determined from one or more IMUs in the insole. In order to further improve accuracy, outlier measurements can be omitted. For example, values that are more than 3 degrees Celsius different from the previous and/or next day's daily average may be omitted.

In order to determine the phase of the user's menstrual cycle, changes in the daily average temperature over the multi-day period may be identified. For example, the daily average temperature may be tracked to identify a temperature rise or drop that is indicative of the ovulation phase or menstruation phase of the user's menstrual cycle. In particular, for users with a biphasic menstrual cycle, a rise in daily average temperature of 0.5 to 0.8 degrees Celsius over a period of 2 days can indicate that the ovulation phase is imminent. Further, a subsequent drop (e.g. after 14 days) indicates that the menstruation phase is imminent. Alternatively, for users with a monophasic menstrual cycle, a gradual increase in the daily average temperature may indicate that ovulation has occurred. Alternatively, for users with a triphasic menstrual cycle, an additional increase in the daily average temperature may indicate that the menstruation phase is imminent.

The type of menstrual cycle (i.e. biphasic, monophasic, or triphasic) may be determined by monitoring the daily average temperature over a time period that is extended (e.g. several months), and identifying patterns in the daily average temperatures. Furthermore, patterns may be identified in time-point average temperatures.

Optionally, in order to aid in pattern recognition, the user may input information such as a first date of menstruation, the date that menstrual cycle symptoms occurred, or a date that is believed to be the date of ovulation. This information may be input using the secondary device (e.g. via the user interface 402).

Optionally, after a pattern has been established, predictions may be made. That is, over an extended time period, the measurements can be processed to identify patterns. The patterns can then be used to predict a future phase of a future menstrual cycle, and an indication of the prediction can be output. For example, the user interface 402 may display an indication that in the next menstrual cycle, ovulation will occur on day 14.

Over time, the accuracy of menstrual cycle predictions may be improved. These improvements may be achieved via machine learning. Furthermore, the user may have the opportunity to correct inaccurate predictions. For example, if a prediction is made that menstruation will last for 5 days, but instead it lasts for 7 days, this can be input by the user. This may improve the machine learning.

In a further example, a likelihood of pregnancy may be determined. Particularly, if an elevated temperature persists past the expected date of menstruation, this may indicate pregnancy. The user interface 402 may thus display an indication that pregnancy is a possibility, and instruct the user to take a further pregnancy test and/or contact a doctor.

Now with regards to step 508, as a first example of determining a second health metric, a risk of tissue ulceration may be determined. In particular, the risk of developing a foot ulcer (such as a diabetic foot ulcer) at the first location and/or second location (and so on) may be determined. For example, for individual time points within the time period, the measurement from the first temperature sensor 110a and the measurement from the second temperature sensor 110b may be processed to determine whether the temperature at the first location and the temperature at the second location differ by a predetermined threshold. The predetermined threshold may be, for example, about 4 degrees Celsius. If a temperature at one location is 4 degrees Celsius (or more) higher than a temperature at one or more other locations, this can indicate that tissue ulceration has occurred or is imminent. Alternatively, the threshold may not be predetermined, and instead may be determined on an individual basis (e.g. as described above with regards to setting the fever threshold).

Optionally, as diabetic foot ulcers are known to occur at plantar locations on a user's foot, temperature sensors 110 at plantar locations can be used to determine the risk of tissue ulceration. That is, the first location can be a first plantar location and the second location can be a second plantar location.

Optionally, step 508 can be carried out using a single insole 100. That is, the first and second locations can be on the same foot (i.e. a first foot). Alternatively, step 508 can be carried out using a pair of insoles 100, where the first and second locations are on different feet. That is, the first location can be a first plantar location on a first foot of the user, and the second location can be a second plantar location on the second foot of the user.

Optionally, in addition to processing temperature measurements to determine one or more health metrics (e.g. a risk of tissue ulceration), pressure measurements and/or other sensor measurements may be processed to determine one or more health metrics. For example, pressure measurements may be processed to identify shear forces, which in turn can indicate risk of tissue ulceration. In particular, high pressure measurements in combination with high temperature measurements may indicate likelihood of immediate ulcer, while high pressure measurements and lower temperature measurements may indicate likelihood of future ulcer.

As a second example of determining a second health metric, a location of poor circulation may be determined. This example may be similar to the example described above for determining a risk of tissue ulceration; however, locations of lowered temperature may identified. That is, if a temperature at one location is lower than a temperature at one or more other locations by a threshold (e.g. a predetermined threshold or an individually determined threshold), this can indicate that the location with lower temperature has poor circulation.

An indication of the second health metric can then be output (step 514). For example, the secondary device can display an alert that indicates that tissue ulceration is imminent, or that indicates that poor circulation is occurring.

If it is determined that there is a risk of tissue ulceration (e.g. a high risk or a moderate risk), then the user may be further instructed (e.g. via the user interface 402) to offload the relevant foot in order to minimize the risk of tissue ulceration, and/or to contact a health care practitioner. Likewise, if it is determined that there is a location of poor circulation, then the user may be further instructed (via the user interface 402) to contact a health care practitioner.

Various further first health metrics and second health metrics may be determined. For example, temperature changes from heartbeat to heartbeat may be identified, and/or calorie burn may be determined. Further examples of health metrics include circadian rhythm, meal digestion, super-imposed circadian rhythm and menstrual cycle rhythms, changes in hormone levels (e.g. to track health outcomes in individuals receiving hormone therapy, such as for gender transitioning), response to a medication (e.g. if a user is put on a new medication, the systems and processes described herein may be used to monitor the user before use and during use of the new medication, to provide an indication of how their body may be reacting and if they are experiencing any side effects) and/or palpable pulse in different foot regions to indicate blood flood).

In some examples, both a first and a second health metric can provide information about the same health concern. For example, to identify poor circulation, both a first and second health metric can be determined. For the first health metric, a drop in limb temperature over a time period may be identified (e.g. when someone exits their warm house to go outside in the winter), and then the second health metric can include a comparison of the limb temperature to the temperature of a different limb. Both metrics can be used to identify poor circulation.

In any of the above examples, machine learning may be used to update thresholds and/or algorithms, in order to improve health metric determination (e.g. to improve accuracy and/or pattern recognition).

In any of the above examples, the user may input various information, to aid the system in determining health metrics. For example, in instances where the wearable device is an insole, the user may input information relating to whether they are wearing a sock, and the system may then correct the temperature measurements based on this information. Further examples of information that may be input by the user include whether the user is taking any medication (e.g. hormonal contraceptives), the type of footwear being worn, and/or information relating to pain (e.g. a foot pain rating).

The devices, systems, and processs described above may have various uses, such as in healthcare, athletics, occupational health and safety, rehabilitation, and the military. For example, the insole 100 may be used by military personnel taking part in a training exercise extending over several days. The insole 100 may be used to ensure personnel remain safe and healthy during the training exercise. Particularly, temperature may be tracked to determine risk of pressure wounds, hypothermia, or hyperthermia, and to detect early signs of illness or even some injuries.

While the above description provides examples of one or more processes or apparatuses or compositions, it will be appreciated that other processes or apparatuses or compositions may be within the scope of the accompanying claims.

To the extent any amendments, characterizations, or other assertions previously made (in this or in any related patent applications or patents, including any parent, sibling, or child) with respect to any art, prior or otherwise, could be construed as a disclaimer of any subject matter supported by the present disclosure of this application, Applicant hereby rescinds and retracts such disclaimer. Applicant also respectfully submits that any prior art previously considered in any related patent applications or patents, including any parent, sibling, or child, may need to be re-visited.

We claim:

1. A process for using temperature measurements to determine health metrics, comprising:
a. while a wearable device is worn by a user, using a first temperature sensor of the wearable device to obtain a measurement at a first location on the user's body;
b. serially repeating step a. to obtain a first set of measurements over a time period;
c. using a second temperature sensor of the wearable device to obtain a measurement at a second location on the user's body;
d. serially repeating step c. to obtain a second set of measurements over the time period;
e. processing at least some measurements of the first set of measurements and/or at least some measurements of the second set of measurements to identify changes in a body temperature of the user over the time period, determining at least one first health metric based on the changes, and outputting an indication of the first health metric; and
f. for each of a plurality of time-points within the time period, processing the measurement of the first set taken at the time-point and the measurement of the second set taken at the time-point to compare the temperature at the first location at the time point to the temperature at the second location at the time point, determining at least one second health metric based on the comparison, and outputting an indication of the second health metric.

2. The process of claim 1, wherein the first health metric comprises a phase of a menstrual cycle of the user.

3. The process of claim 2, wherein the time period is a multi-day period, and step e. comprises:
i. for each day in the multi-day period, determining a daily average temperature by processing a subset of the first set of measurements and a subset of the second set of measurements, wherein the subset of the first set of measurements and the subset of the second set of measurements are taken from a daily window; and
ii. identifying changes in the daily average temperature over the multi-day period to identify the phase of the menstrual cycle of the user, and outputting an indication of the phase of the menstrual cycle.

4. The process of claim 3, wherein step ii. comprises identifying an ovulation phase and/or a menstruation phase in the menstrual cycle of the user, and the process further comprises predicting a future phase of a future menstrual cycle, and outputting an indication of the prediction.

5. The process of claim 3, further comprising:
i. using the wearable device to identify a time period during which the user is at rest; and
ii. setting the daily window as the time period during which the user is at rest.

6. The process of claim 1, wherein step e. comprises:
i. for each of the plurality of time-points within the time period, determining a time-point average temperature by processing the measurement of the first set at the time-point and the measurement of the second set at the time-point; and
ii. identifying changes in the time-point average temperatures over the time period to identify the first health metric, and outputting the indication of the first health metric.

7. The process of claim 6, wherein the first health metric comprises a period of physical activity for the user, and step ii. comprises identifying an increase in the time-point average temperature over the time period to identify the period of physical activity for the user.

8. The process of claim 6, wherein the first health metric comprises a fever state for the user, and step ii. comprises identifying whether the time-point average temperature has exceeded a fever threshold over the time period to identify the fever state of the user.

9. The process of claim 1, wherein the second health metric comprises a risk of tissue ulceration at the first location and/or the second location, and step f. comprises identifying whether the temperature at the first location differs from the temperature at the second location by a predetermined threshold.

10. The process of claim 1, wherein the wearable device comprises an insole or a pair of insoles, the first location is a first plantar location on a first foot of the user, and the second location is a second plantar location on the first foot of the user or the second foot of the user.

11. A process for using temperature measurements to determine health metrics, comprising:
a. while a wearable device is worn by a user, using a first temperature sensor of the wearable device to obtain a measurement at a first location on the user's body;
b. serially repeating step a. to obtain a first set of measurements over a time period;
c. using a second temperature sensor of the wearable device to obtain a measurement at a second location on the user's body;
d. serially repeating step c. to obtain a second set of measurements over the time period;
e. for each of a plurality of time-points within the time period, determining a time-point average temperature by processing the measurement of the first set at the time-point and the measurement of the second set at the time-point;
f. identifying changes in the time-point average temperatures over the time period to identify at least one first health metric for the user, and outputting an indication of the first health metric; and
g. for each of the plurality of time-points within the time period, processing the measurement of the first set taken at the time-point and the measurement of the second set taken at the time-point to compare the temperature at the first location at the time point to the temperature at the second location at the time point, determining at least one second health metric based on the comparison, and outputting an indication of the second health metric.

12. The process of claim 11, wherein the first health metric comprises a period of physical activity for the user, and step f. comprises identifying an increase in the time-point average temperatures over the time period to identify the period of physical activity for the user.

13. The process of claim 11, wherein the first health metric comprises a fever state for the user, and step f. comprises identifying whether the time-point average temperatures have exceeded a fever threshold over the time period to identify the fever state of the user.

14. The process of claim 13, further comprising instructing the user to take a COVID-19 test if the time-point average temperatures have exceeded a fever threshold over the time period

15. The process of claim 11, wherein the second health metric comprises a risk of tissue ulceration at the first location and/or the second location, and step g. comprises identifying whether the temperature at the first location differs from the temperature at the second location by a threshold.

16. The process of claim 11, wherein the wearable device comprises an insole or a pair of insoles, the first location is a first plantar location on a first foot of the user, and the second location is a second plantar location on the first foot of the user or a second foot of the user.

17. A process for using temperature measurements to determine health metrics, comprising:
a. while a wearable device is worn by a user, using a first temperature sensor of the wearable device to obtain a measurement at a first location on the user's body;
b. serially repeating step a. to obtain a first set of measurements over a multi-day time period;
c. using a second temperature sensor of the wearable device to obtain a measurement at a second location on the user's body;
d. serially repeating step c. to obtain a second set of measurements over the multi-day time period;
e. for each day in the multi-day period, determining a daily average temperature by analyzing a subset of the first set of measurements and a subset of the second set of measurements, wherein the subset of the first set of measurements and the subset of the second set of measurements are taken from a daily window;
f. identifying changes in the daily average temperatures over the multi-day period to identify at least one first health metric for user, and outputting an indication of the first health metric; and
g. for each of a plurality of time-points within the multi-day period, processing a measurement of the first set taken at the time-point to a measurement of the second set taken the time-point to compare the temperature at the first location at the time point to the temperature at the second location at the time point, determining at least one second health metric based on the comparison, and outputting an indication of second health metric.

18. The process of claim 17, wherein the first health metric comprises a phase of a menstrual cycle of the user, and step f. comprises identifying an ovulation phase and/or a menstruation phase in the menstrual cycle of the user.

19. The process of claim 18, further comprising:
i. using the wearable device to identify a time period during which the user is at rest; and
ii. setting the daily window as the time period during which the user is at rest.

20. The process of claim 17, wherein the second health metric comprises a risk of tissue ulceration at the first location and/or the second location, and step g. comprises identifying whether the temperature at the first location differs from the temperature at the second location by a predetermined threshold.

21. The process of claim 17, wherein the wearable device comprises an insole or a pair of insoles, the first location is a first plantar location on a first foot of the user, and the second location is a second plantar location on the first foot of the user or a second foot of the user.
